(19) United States
(12) Patent Application Publication
(10) Pub. No.: US 2003/0180952 A1
(43) Pub. Date: Sep. 25, 2003

(54) Plant regeneration

(75) Inventors: Prakash Lakshmanan, Jamboree Heights (AU); Adrian Ross Elliott, Auchenflower (AU); Grant Richard Smith, Bellowrie (AU); Robert Jason Geijskes, Kenmore (AU); Kerry Anne Nutt, Indooroopilly (AU); Christopher Peter Leslie Grof, The Gap (AU); Nils Berding, Bayview Heights (AU)

Correspondence Address: MORGAN, LEWIS & BOCKIUS LLP, 1701 MARKET STREET, PHILADELPHIA, PA, 19103-2921, US

(73) Assignee: Sugar Research & Development Corporation

(21) Appl. No.: 10/278,448

(22) Filed: Oct. 23, 2002

Related U.S. Application Data
(63) Continuation of application No. PCT/AU01/00438, filed on Apr. 27, 2001.

(30) Foreign Application Priority Data
Apr. 28, 2000 (AU) PQ 7199
Nov. 10, 2000 (AU) PR 1430

(51) Int. Cl. A01H 5/00; C12N 15/82

(52) U.S. Cl. 435/468; 800/320; 800/320.3

(57) ABSTRACT

A method of plant micopropagation is provided whereby a thin section of non-callus tissue obtained from a monocotyledonous plant, such as sugarcane, wheat or sorghum, is cultured in the presence of a cytokinin and/or an auxin. Optimal regeneration occurs when a basal surface of the thin section is oriented so as to be substantially not in contact with the culture medium. This micropropagation method produces mature monocotyledonous plants by either organogenic or embryogenic regeneration without substantial callus formation. By avoiding use of callus as a starting tissue, this method reduces the likelihood of propagated plants displaying somaclonal variation.

### FIELD OF THE INVENTION

THIS INVENTION relates to a method of regenerating monocotyledonous plants. In particular, this invention applies to a micropropagation method for directly regenerating plants of the Graminae family, such as sugarcane and cereals, although without being limited thereto.

### BACKGROUND OF THE INVENTION

Plant tissue culture has been used extensively in plant propagation, transformation, mutagenesis, breeding and virus elimination. Such tissue culture systems are generally referred to as “micropropagation” systems, wherein plant tissue explants are cultured in vitro in a suitable solid or liquid medium, from which mature plants are regenerated.

However, a persistent problem inherent in plant regeneration systems is somaclonal variation. Somaclonal variation often results in reduced agronomic performance of regenerated plants compared with the plant(s) from which they are derived. Although, in principle, this problem can be overcome by backcrossing, in certain situations such as generation of transgenic sugarcane, it is generally desirable to retain the elite characteristics of the variety or cultivar, without further manipulation such as backcrossing.

This problem of somaclonal variation is particularly evident with callus-based regeneration techniques, which are commonly used in plant regeneration systems. The advantage of callus is that it has proven to be a useful tissue obtainable from a wide variety of plants for the purposes of regeneration and transformation. However, the relatively uncontrolled cell proliferation in callus provides considerable potential for genetic variation in plants generated therefrom.

Thus, in the interests of reducing somaclonal variation, a considerable amount of effort has been aimed at identifying alternative plant tissues suitable for the purposes of plant propagation and transformation. In this regard, meristematic tissue has been shown to exhibit lower somaclonal variation than callus (Irvine et al., 1991, Plant Cell Tissue Organ Cult. 26 15), and can be used as a basis for plant propagation. Such non-callus based methods are generally referred to as “direct” regeneration methods.

Cytokinins and/or auxins are agents which have often been used to induce direct regeneration from non-callus tissue.

Particular examples of the use of auxins and/or cytokinins in the direct regeneration of monocotyledonous plants may be found in Irvine & Benda, 1987, Sugarcane 6 14, Irvine et al., 1991, Plant Cell Tissue Organ Cult. 26 115, Burner & Grisham, 1995, Crop Sci. 35 875, Alam et al., 1995, Sugarcane 6 20 and Lakshamanan et al., 1996, J. Orch. Soc. Ind. 10 31. With regard to some of these publications, attention is drawn to the efficacy of sectioned explants of non-callus tissue such as leaf sections (Irvine & Benda, 1987, supra; Alam et al., 1995, supra), sections of shoot tips, young leaves, inflorescence and protocorm-like bodies (Lakshamanan et al., 1996, supra), for the purposes of direct regeneration.

Other examples of regeneration from inflorescence include paspalum (Bovo & Mroginski, 1986, J. Plant. Physiol. 124 481), tulips (Taeb & Alderson, 1987, Acta Horticulture 212 677) and sugarcane (Liu, 1993, J. Plant Physiol. 141 714).

Plant tissues such as those described above are often utilized in culture as segments, slices or sections. One particular type of section shown to be useful in plant regeneration is a thin section (TS) explant. For example, thin sections have been studied with respect to the characteristics of shoot generation in tobacco (Kaur-Sawhney et al.,1988, Planta 173 282), poplar (Lee-Stadelmann et al., 1989, Plant Sci. 61 263) and rapeseed (Pihakaski-Maunsbach et al., 1993, Physiologica Plantarum 87 167). Furthermore, thin sections have shown promise for propagating plants such as commercially grown orchids (Lakshamanan et al., 1996, J. Orchid Soc. India 10 31; Lakshamanan et al., 1995, Plant Cell Rep. 14 510), transgenic orchids (Toh et al., 1995, Proceedings 3rd Undergrad. Science Res. Cong. Singapore pp92-98) and mangosteen (Goh et al., 1994, Plant Science 101 173).

With shoot regeneration from thin sections, there has been evidence that the orientation of the thin section during culture can influence regeneration efficiency. This was reported by Slabbert et al., 1995, Plant Cell Tiss. Org. Cult. 43 51, using 2.5-5.0 mm sections of monocot Crinum macowanii (bush lily) immature floral stem, oriented so that their morphologically basal ends were in contact with the culture medium.

### SUMMARY OF THE INVENTION

Despite progress being made with respect to micropropagation of monocotyledonous plants, efficient and reproducible generation of mature monocotyledonous plants displaying minimal somaclonal variation has remained elusive. Surprisingly, the present inventors have discovered that explant orientation during culture is critical to maxmizing the efficiency of in vitro plant regeneration, the orientation being opposite to that taught by Slabbert et al., 1995, supra.

Therefore, in one aspect, the present invention resides in a method of plant micropropagation including the step (i) of culturing an explant from a monocotyledonous plant in a culture medium comprising a cytokinin and/or an auxin wherein a basal surface of said explant is oriented so as to be substantially not in contact with said medium during culture.

Preferably, the method includes the step (ii) of culturing the explant to produce plant shoots having shoot meristems.

Preferably, the method at step (ii) further includes culturing excised shoots to produce a plantlet.

Preferably, the method includes the step (iii) of propagating the plantlet obtained at step (ii) to produce a mature plant.

In another aspect, the invention provides regenerable tissue, plantlets and monocotyledonous plants produced according to the method of the first-mentioned aspect.

Also contemplated are cells, tissues, leaves, fruit, flowers, seeds and other reproductive material, material useful for vegetative propagation, F1 hybrids and all other plants and plant products derivable from said monocotyledonous plant.

Preferably, the monocotyledonous plant is of the Gramineae family which includes sugarcane and cereals such as wheat, rice, rye, oats, barley, sorghum and maize. Other monocotyledonous plants which are contemplated include bananas, lilies, tulips, onions, asparagus, ginger, bamboo, oil palm, coconut palm, date palm and ornamental palms such as kentia and rhapis palms.

More preferably, the monocotyledonous plant is selected from the group consisting of sugarcane, sorghum and wheat.

Throughout this specification, it will be understood that “comprise”, “comprises” and “comprising” are used inclusively rather than exclusively, in that a stated integer or group of integers may include one or more non-stated integers or groups of integers.

### BRIEF DESCRIPTION OF THE FIGURES AND TABLES

FIG. 1: Representative stages of plant regeneration from sugarcane explants micropropagated in vitro. A=sugarcane leaf spindle explants oriented on solid medium (supplememented with an auxin and cytokinin) so that the basal surface is substantially not in contact with medium. B=early shoot meristem growth by plantlet bodies. C=advanced shoot growth by plantlet bodies. D=root growth by plantlet bodies in solid medium without a cytokinin or auxin. E=sugarcane plantlets after transfer to field.

FIG. 2: Schematic depiction of in vitro plantlet regeneration from 1-2 mm explants of sugarcane leaf whorl.

FIG. 3: Regeneration from inflorescence sections. (a) Shoots/plants regenerating from a single cultured thin section of inflorescence. (b) Shoots/plants regenerating from cultured thin sections of inflorescence tissue. (c) Shoots/plants regenerating on the main floral stem isolated from a cultured thin section of floral tissue. (d) High magnification view of shoots/plants regenerating from pieces of thin sections of inflorescence tissue after 69 days culture. A root can be seen growing upwards from the central plant. (e) high magnification view of a thin section of inflorescence tissue after 2 days culture.

FIG. 4: Plantlet regeneration from thin section explants (leaf base) of sorghum variety “New Nugget”.

FIG. 5: Well developed sorghum plants regenerated from thin section explants such as shown in FIG. 4.

FIG. 6: Direct regeneration of wheat plants from thin transverse sections of wheat “stems”. Examples shown here were cultured in the presence of 10 μM CPA for 21 days before transfer to 5 μM zeatin. (a) swelling of thin section explants (b) shoots regenerating on the small explants (c) visible green shoots and roots (d) and (e) shoots forming at a later stage (f) a number of small shoots on an explant and (g) a regenerated wheat plant in culture.

Table 1: Influence of explant size and orientation on shoot production in leaf explants of sugarcane cultivar Q165 after 5 weeks of culture on MS medium containing 4 μM 6-benzyladenine (BA) and 10 μM α-napthaleneacetic acid (NAA). Fifteen to twenty replicates, each with 6 to 10 explants were maintained for each treatment. “Top down” corresponds to basal surface substantially not in contact with medium; “top up” refers to basal surface in contact with medium.

Table 2: Influence of explant size and orientation on shoot production in leaf explants of sugarcane cultivar Q165 after 6 weeks of culture on MS medium containing 4 μM 6-benzyladenine (BA) and 10 μM α-napthaleneacetic acid (NAA). Ten to 15 replicates, each with 5 to 10 explants were maintained for each treatment. “Top down” corresponds to basal surface substantially not in contact with medium; “top up” refers to basal surface in contact with medium.

Table 3: Influence of explant orientation on shoot regeneration in 1-2 mm and 5-6 mm thick leaf segments of sugarcane cultivar Q165 after 8 weeks of culture on MS medium containing 4 μM 6-benzyladenine (BA) and 10 μM α-napthaleneacetic acid (NAA). Five replicates, each with 5 explants, were cultured for each treatment. “Top down” corresponds to basal surface substantially not in contact with medium; “top up” refers to basal surface in contact with medium.

Table 4: Spatial distribution of regeneration response in the leaf spindle of sugarcane cultivar Q165. Thirty serial leaf sections (1-2 mm thick) were prepared from leaf spindle, beginning just above the shoot meristem region, divided them into three groups (the 10 lower most segments: basal; the next 10 segments: middle; the uppermost 10 segments: apical), and cultured each group separately on MS medium supplemented with 6-benzyladenine (BA) and α-napthaleneacetic acid (NAA) for 8 weeks. Five replicates, each with 10 explants placed in top down orientation, were maintained for each treatment.

Table 5: Spatial distribution of regeneration response in the leaf spindle of sugarcane cultivar Q165. Thirty serial leaf sections (1-2mm thick) were prepared from leaf spindle, beginning just above the shoot meristem region, divided them into three groups (the 10 lower most segments: basal; the next 10 segments: middle; the uppermost 10 segments: apical), and cultured each group separately on MS medium supplemented with 6-benzyladenine (BA) and α-napthaleneacetic acid (NAA) for 11 weeks. Four to 5 replicates, each with 10 explants placed in top down orientation, were kept for each treatment.

Table 6: Spatial distribution of shoot regeneration response in the leaf spindle of sugarcane cultivar 90N876. Thirty serial leaf sections (1-2 mm thick) were prepared from leaf spindle, beginning just above the shoot meristem region, divided them into three groups (the 10 lower most segments: basal; the next 10 segments: middle; the uppermost 10 segments: apical), and cultured each group separately on MS medium supplemented with 6-benzyladenine (BA) and α-napthaleneacetic acid (NAA) for 6 weeks. Five replicates, each with 10 explants cultured in top down orientation, were maintained for each treatment.

Table 7: Relative efficiency of 6-benzyladenine (BA) and kinetin (KIN) on shoot induction in leaf sections (1-2 mm) of sugarcane cultivar Q165 after 7 weeks of culture. Twenty to 25 replicates, each with 10 explants, were maintained for each treatment. Explants were not placed to any particular orientation on the culture medium.

Table 8: Effect of different levels of 6-benzyladenine (BA) and α-napthaleneacetic acid (NAA) on shoot production in leaf sections (1-2 mm thick) of sugarcane cultivar Q165 after 8 weeks of culture. Ten to 14 replicates, each with 10 explants placed in top down orientation were maintained for each treatment.

Table 9: Effect of different levels of α-napthaleneacetic acid (NAA) on shoot regeneration in leaf sections (1-2 mm thick) of sugarcane cultivar Q165 cultured for 8 weeks on 6-benzyladenine (BA) and NAA enriched-media. Fifteen to 20 replicates, each with 10 explants, were maintained for each treatment. Explants were cultured in top down orientation.

Table 10: Shoot regeneration in 5-6 mm thick leaf segments of sugarcane cultivar Q165 after 11 weeks of culture on MS medium supplemented with different amounts of 6-benzyladenine (BA) and α-napthaleneacetic acid (NAA). Five to 6 replicates, each with 5 explants placed in top down orientation, were maintained for each treatment.

Table 11: Direct shoot regeneration in leaf explants (1-2 mm thick) of sugarcane cultivar Q179 cultured on MS medium supplemented with 6-benzyladine (BA) and α-napthaleneacetic acid (NAA) for 5 weeks. Ten replicates, each with 10 explants cultured in top down orientation, were maintained for each treatment.

Table 12: Direct shoot regeneration in leaf explants (1-2 mm thick) of sugarcane cultivar 90N876 cultured on MS medium supplemented with 6-benzyladenine (BA) and α-napthaleneacetic acid (NAA) for 8 weeks. Thirty replicates, each with 10 explants cultured in top down orientation, were maintained for each treatment. Table 13: Direct shoot regeneration in leaf explants (1-2 mm thick) of sugarcane cultivar 91N406 cultured on MS medium supplemented with 6-benzyladenie (BA) and α-napthaleneacetic acid (NAA) for 8 weeks. Five replicates, each with 10 explants cultured in top down orientation were maintained for each treatment.

Table 14: Direct shoot regeneration in leaf explants (1-2 mm thick) of sugarcane cultivar Q187 cultured on MS medium supplemented with 6-benzyladenine (BA) and α-napthaleneacetic acid (NAA) for 6 weeks. Twelve to 16 replicates, each with 10 explants cultured in top down orientation, were maintained for each treatment.

Table 15: Direct shoot regeneration in 1-2 mm thick leaf sections of sugarcane cultivar Q124 after 11 weeks of culture on MS medium supplemented with different amounts of 6-benzyladenine (BA) and α-napthaleneacetic acid (NAA). Five to 6 replicates, each with 10 explants placed in top down orientation, were maintained for each treatment.

Table 16: Direct shoot regeneration in 1-2mm thick leaf explants of sugarcane cultivar Q57 and Q117 after 6 weeks of culture on MS medium containing 6-benzyladenine (BA) and α-napthaleneacetic acid (NAA). Five to 10 replicates, each with 10 explants placed in top down orientation, were maintained for each treatment.

Table 17: Analysis of variance of number of tillers per stool for sugarcane cultivars Q96 and Q 117 established from one-eye setts and plantlets derived from cultured transverse section of spindle rolls.

Table 18: Comparison of regeneration efficiency for different sugarcane explants. Explants were (a) 2-3 mm sections of main floral axis of inflorescence; (b) ˜8 mm sections of main floral axis placed laterally on the culture medium; (c) immature floral tissues dissociated from floral axis; and (d) whole 2-3 mm sections of inflorescence oriented “top down”. B4N10=4 μM BA+10 μM NAA; B4N40=4 μM BA+40 μM NAA; B4N60=4 μM BA+60 μM NAA; 2,4D14=14 μM 2,4,D. “Top down” orientation is as previously described. Shoot frequency legend: *1-2 shoots per explant; **3-10 shoots per explant; ***greater than 25 shoots per explant.

Table 19: Morphogenic response of leaf whorl explants (1.0-2.0 mm thick) of sugarcane cultivar Q165 after 8 weeks of culture on MS medium supplemented with chlorophenoxyacetic acid (CPA). Ten replicates, each with 10 explants placed in top down orientation, were maintained for each treatment. Explants were cultured under dark condition for the initial 5-6 weeks and then transferred to light (16 hr/day) for further shoot/planlet development. C=Mostly non-embroygenic callus was produced.

Table 20: Morphogenic response of leaf whorl explants (1.0-2.0 mm thick) of sugarcane cultivar Q165 after 8 weeks of culture on MS mediun supplemented with chlorophenoxyacefic acid (CPA). Ten replicates, each with 10 explants placed in top down orientation, were maintained for each treatment. All cultures received 16 hr light every day.

Table 21: Shoot regeneration in 1.0-2.0 mm thick leaf whorl explants of sugarcane cultivar Q165 after 8 weeks of culture on MS medium containing 6-benzyladenine (BA), α-naphthaleneacetic acid (NAA) and cholorophenoxyacetic acid (CPA). Ten replicates, each with 10 explants placed in top down orientation, were maintained for each treatment. All cultures received 16 hr light every day.

Table 22: Shoot regeneration in 1.0-2.0 mm thick leaf explants of sugarcane cultivar Q165 after 8 weeks of culture on MS medium containing 6-benzladenine (BA), α-naphthaleneacetic acid (NAA) and 3 amino-2,5-dichlorobenzoic acid (AD). Ten replicates, each with 10 explants placed in “top down” orientation, were maintained for each treatment. All cultures received 16 hr light every day.

Table 23: Shoot regeneration in 1.0-2.0 mm thick leaf explants of sugarcane cultivar 85C542 after 8 weeks of culture on MS medium containing 6-benzyladenine (BA), α-naphthaleneacetic acid (NAA) and cholorophenoxyacetic acid (CPA). Ten replicates, each with 10 explants placed in “top down” orientation, were maintained for each treatment. All cultures received 16 hr light every day.

Table 24: Shoot regeneration in 1.0-2.0 mm thick leaf explants of sugarcane cultivar 85C542 after 8 weeks of culture on MS medium containing 6-benztladenine (BA), α-naphthaleneacetic acid (NAA) and 3 amino-2,5-dichlorobenzoic acid (AD). Ten replicates, each with 10 explants placed in “top down” orientation, were maintained for each treatment. All cultures received 16 hr light every day. * Only a few shoots were formed in each regenerating explant; C=Only callus production occurred.

Table 25: Shoot regeneration in 1.0-2.0 mm thick leaf explants of sugarcane cultivar 85C542 after 8 weeks of culture on MS medium containing 6-benzyladenine (BA), α-naphthaleneacetic acid (NAA) and 3 amino-2,5-dichlorobenzoic acid (AD). Ten replicates, each with 10 explants placed in “top down” orientation, were maintained for each treatment. Explants were cultured under dark condition for the initial 5-6 weeks and then transferred to light (16 hr/day) for further shoot/planlet development.

Table 26: Effect of different levels of cholorophenoxyacetic acid (CPA) on somatic embryogenesis and shoot production in 1.0-2.0 mm thick leaf base sections of sorghum (Sorghum bicolor L.) variety New Nugget after 8 weeks of culture. Five to six replicates, each with 10 explants placed in “top down” orientation, were maintained for each treatment. All cultures received 16 hr light every day.

Table 27: Effect of different levels of cholorophenoxyacetic acid (CPA) and 6-benzyladenine (BA) on somatic embryogenesis and shoot production in 1.0-2.0 mm thick leaf base sections of sorghum (Sorghum bicolor l L.) variety New Nugget after 8 weeks of culture. Five to six replicates, each with 10 explants placed in “top down” orientation, were maintained for each treatment. All cultures received 16 hr light every day.

Table 28: Regeneration of wheat shoots and plants from thin transverse stem sections placed on media with different phytohormone treatments. Transverse “stem” sections were prepared from wheat plants 15-40 cm high. Thin transverse sections were taken from regions of the “stems” (leaf whorls) of wheat plants below the young floral (internal) primordia and from regions surrounding and above the young floral primordia. The sections were 0.3-0.8 cm in diameter and up to 2.0 mm thick. Explants were placed with the apical surface contacting the culture medium (“top down”; a and b) or apical surface “up” (c) and regeneration was scored after twelve weeks. Some treatments solely consisted of twelve weeks culture under a particular phytohormone regime (a and c) whilst in other treatments, explants were cultured on an initial phytohormone treatment regime and then transferred after three weeks to culture medium comprising 5 μM zeatin (b). P=picloram; Z=zeatin; D=dicamba; C=chlorophenoxyacetic acid; B=6 benzyladenine; N=α-napthaleneacetic acid; DP=dichlorophenoxyacetic acid. Whole plants were obtained from (b) C10→Z5 and C40→Z5.

### DETAILED DESCRIPTION OF THE PREFERRED EMBODIMENTS

The present invention is predicated, at least in part, on the discovery that the orientation of an explant of a monocotyledonous plant on a culture medium influences shoot regeneration during culture. More particularly, a basal surface of said explant must be substantially not in contact with said medium to ensure maximal shoot regeneration. As will be described in detail hereinafter, the frequency of explants producing shoots is increased as is the number of shoots produced per explant, when the explant is oriented during culture so that the basal surface is substantially not in contact with the culture medium. This “polarity effect” is also manifested by preferential shoot growth from explants taken distal to the direction of meristematic growth (i.e non-apical sections). It will also be demonstrated that plants regenerated according to the method of the invention exhibit minimal somaclonal variation in the field. Furthermore, the method of the invention is applicable to a number of different plant tissues, including leaf spindle and inflorescence.

More particularly, the present invention provides a method of plant micropropagation for regenerating plants without transition through a lengthy or substantial callus phase. This non-callus regeneration may be organogenic or embryogenic.

Suitably, the explant is obtained from plant tissues including leaf spindle or whorl, leaf blade, axillary buds, stems, shoot apex, leaf sheath, internode, petioles, flower stalks, root or inflorescence. A relevant biological property of such suitable tissues is that they contain actively dividing cells having growth and differentiation potential.

Preferably, the explant is obtained from leaf spindle or whorl, or from inflorescence.

With regard to sugarcane inflorescence, a preferred source is immature inflorescence in the process of bolting to flower. Typically, sections of inflorescence comprise a main floral axis or stem surrounded by immature rachis branches bearing immature floral buds.

Suitably, the explant is a segment, slice or section of plant tissue.

Preferably, the explant is a thin section (TS) explant.

As used herein, a “TS explant” is a plant tissue section 1.0-10.0 mm in thickness or preferably 1.0-6.0 mm in thickness.

Examples of preferred TS explant thicknesses are 1.0-2.0, 2.0-3.0 or 5.0-6.0 mm, depending on the plant and the tissue source of the explant.

As used herein, a “basal surface” of said explant is the surface of said explant distal to the direction of shoot growth of said tissue in an intact plant and proximal to the root system. For example, in the case of sugarcane leaf spindle or inflorescence, the basal surface of the explant is proximal to the apical meristem of the leaf shoot from which the explant is taken. In other words, the basal surface was proximal to the sugarcane stalk in the intact plant.

As used herein, “substantially not in contact with the culture medium” in the context of the orientation of a basal surface of an explant during culture, means that at least the majority of the basal surface (as hereinbefore defined) does not directly contact the culture medium. This definition includes situations where the explant is cultured with an apical surface in direct contact with the culture medium, in which case the basal surface is oriented distally to the culture medium. This definition also includes cases where the explant is placed lengthways horizontally on the medium and neither the basal nor apical surfaces directly contact the medium, except perhaps a portion of the perimeter of each surface which may directly contact the medium.

The explant may be cultured for 5 to 8 weeks. However, as will be appreciated by the skilled person, the culture period can readily be shortened or lengthened as required.

The culture medium may include Murashige & Skoog (MS) nutrient formulation (Murashige & Skoog, 1962, Physiologia Plantarum 15 473) or Gamborg's medium (Gamborg et al., 1968, Exp. Cell. Res 50 151). Preferably, the medium comprises MS formulation. It will be appreciated that the abovementioned media are commercially available, as are other potentially useful media.

The medium may further comprise sucrose, preferably at a concentration of 30 g/L. The medium may additionally include agar, preferably at a concentration of 7.5 g/L. Thus, it will be appreciated that the TS explant may be cultured in solid or liquid medium.

Additional components of the medium are selected from the group consisting of citric acid (CA) and ascorbic acid (AA). Preferably, the concentration of CA in the medium is 100-200 mg/L, or more preferably 150 mg/L. Preferably the concentration of AA in the medium is 50-200 mg/L, or more preferably 100 mg/L

Preferably, the cytokinin is selected from the group consisting of 6-benzyladenine (BA), kinetin (KIN), zeatin, α-isopentyladenosine and diphenylurea.

Preferably, the auxin (or an auxin-like compound) is selected from the group consisting of α-napthaleneacetic acid (NAA), indole-3-butyric acid (IBA), indole-3-acetic acid (IAA), 2,4,5-trichlorophenoxyacetic acid, phenylacetic acid, piclorarn, β-napthoxyacetic acid, dicamba, trans-cinnamic acid, 3 amino-2,5-dichlorobenzoic acid (AD) and p-chlorophenoxyacetic acid (CPA).

Preferably, the cytokinin is present in the culture medium at a concentration in the range 0-20 μM.

Preferably, the auxin is present in the culture medium at a concentration in the range 0-100 μM.

In embodiments where sugarcane is to be regenerated, a preferred cytokinin in the culture medium is BA or KIN.

Preferably, BA or KIN is present at a concentration in the range 4-12 μM.

Advantageously, the concentration is 4 μM.

For the specific purpose of organogenic sugarcane regeneration, a preferred auxin in the culture medium is NAA.

Preferably, NAA is present at a concentration in the range 10-60 μM.

More preferably, NAA is present at a concentration of 10 μM.

In cases where sugarcane regeneration is to occur via somatic embryogenesis, the preferred auxin is CPA.

Preferably, CPA is present in the culture medium at a concentration in the range 5-10 μM.

Additionally, NAA and BA may be present in the culture medium at preferred concentrations of 10 μM and 4 μM respectively.

In embodiments of the method relating to cereals such as sorghum, the preferred auxin is CPA.

Preferably, CPA is present at a concentration in the range 4-40 μM.

The preferred cytokinin in such cases is BA at a preferred concentration of 2.5 μM, although it should be understood that the presence of BA is optional.

Preferably, after regeneration is initiated, kinetin is added to the culture medium (with a reduced concentration of CPA, for example 1-2 μM) at a preferred concentration of 2 μM.

In embodiments of the method relating to cereals such as wheat, the preferred auxin is CPA.

A preferred concentration is in the range 5-40 μM.

Preferably, there is no cytokinin present initially during step (i).

However, as will be described in detail hereinafter, the cytokinin zeatin may be included later once regeneration has initiated, at a preferred concentration of 5 μM.

Preferably, zeatin is included in the absence of CPA.

It should also be noted that 3 amino-2,5-dichlorobenzoic acid (AD) is another auxin particularly useful according to the invention, as will be described in more detail hereinafter in the Examples.

Propagation at step (ii) preferably involves two stages:

(a) culturing the explant in the presence of an auxin and/or cytokinin to produce plant shoots having shoot meristems; and

(b) excising said plant shoots and culturing same to produce plantlets having roots.

With regard to (a), the cytokinin and/or auxin used may be the same as, or different to, the cytokinin and/or auxin intially used for culturing the explant.

Preferably, at stage (b) plantlets are propagated in full-strength or half-strength MS medium in the absence of an auxin and/or cytokinin.

As will be evident in the Examples, the addition or replacement of cytokinins and/or auxins at stage (ii) can be tailored according to the plant type and purpose of regeneration.

The plantlets (preferably at 5-10 cms in length) are then propagated in soil or a soil substitute to promote growth into a mature plant. Preferably, propagation of plants from plantlets at step (iii) is performed in Perlite, peatmoss and sand (1:1:1) under glasshouse conditions.

So that the present invention may be readily understood and put into practical effect, the skilled person is referred to the following non-limiting examples.

### EXAMPLE 1

### General Materials and Methods

1.1 Plant Materials

Young developing leaves of sugarcane varieties Q57, Q96, Q117, Q124, Q165, Q187, 85C542, 90N876 and 91N406 were used variously throughout the experiments.

1.2 Preparation of Thin Section Leaf Spindle Explants

Top portions (about 30 cm) of shoots were cut and all leaves except the innermost 3-4 whorls were carefully removed under aseptic conditions. TS explants measuring about 1.0-2.0 mm or 5.0-6.0 mm in thickness were prepared by serial transverse sectioning of the lowermost 3-4 cm portion (just above the apical meristem) of the leaf spindle using a sharp surgical blade under sterile conditions.

1.3 Preparation of Thin Section Inflorescence Explants

Young bolting inflorescence “stalk” tissue enveloped in developing leaves (floral and leaf components) was harvested from above the apical meristem of Q152 or Q165 sugarcane plants. Transverse sections were made to produce 2-3 mm thin sections of the tissue.

1.4 Media and Culture Conditions

Murashige & Skoog (MS) nutrient formulation supplemented with 30 g/L sucrose and 7.5 g/L Difco agar were used as the basal culture medium. Basal medium was enriched with different concentrations and combinations of (i) a cytokinin: 6-benzylaminopourine (BA) or kinetin (KIN); (ii) an auxin: α-napthaleneacetic acid (NAA); and (iii) anti-oxidants: citric acid (CA), ascorbic acid (AA), or dithiothreitol (DTT); depending on the experimental objective. The pH of the medium was adjusted to 5.7 before autoclaving for 20 minutes at 120° C. TS explants were cultured in various orientations either in tissue culture dishes (90×14 mm) with 40 ml agar-solidified medium or in a 100 ml baby food jar containing 40 ml liquid medium, or on membrane rafts with flotation kept in a polypropylene container with 40 ml liquid medium. Liquid cultures were agitated continuously on a gyratory shaker at 120 rpm. All cultures were incubated at 25-28° C. under 16 hr photoperiod provided by cool, white fluorescent tubes. Subculturing was carried out at least once a week, or more frequently if medium or TS turned brown due to phenolic exudation.

1.5 Plant Propagation

Shoots (2-3 cm long) were excised and cultured on either full-strength or half-strength MS medium for root production and further development. Well rooted plantlets were transferred to plastic pots containing Perlite, peatmoss and sand (1:1:1) and hardened under glasshouse conditions.

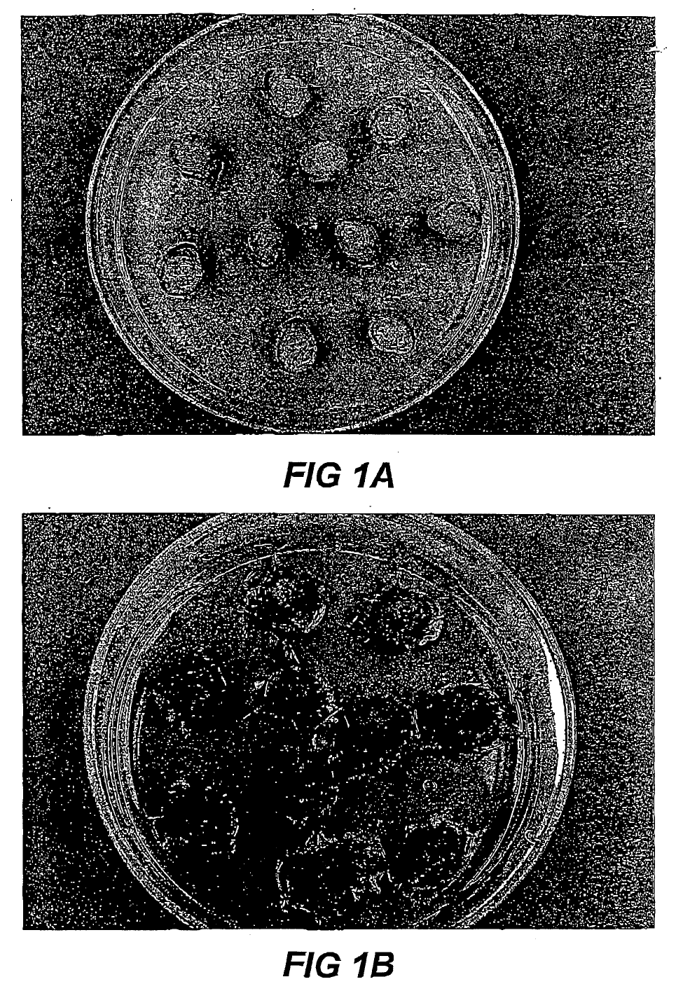

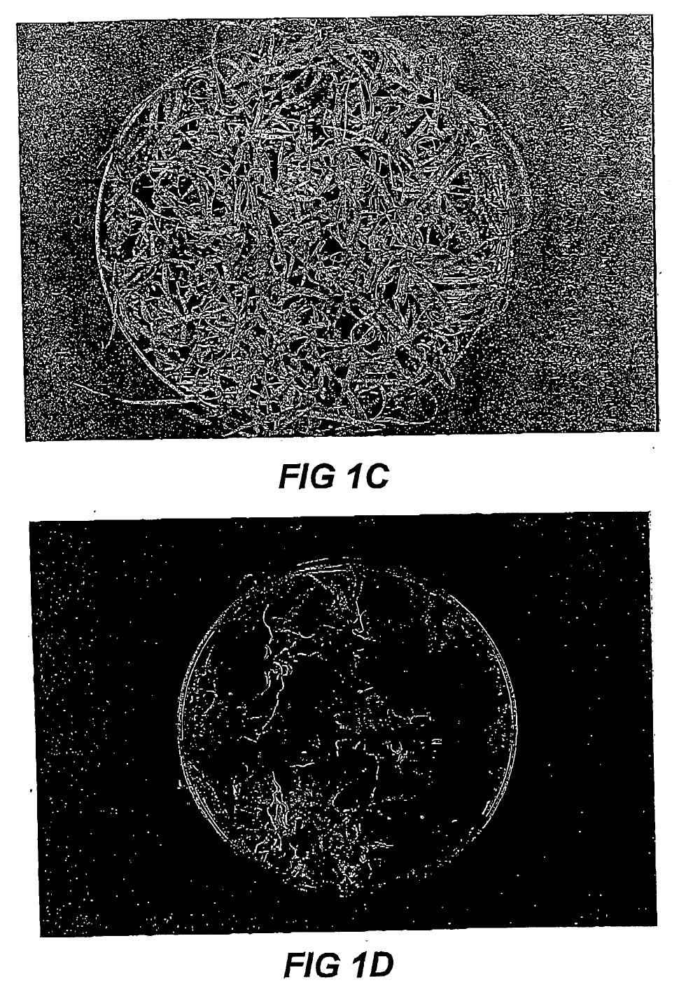

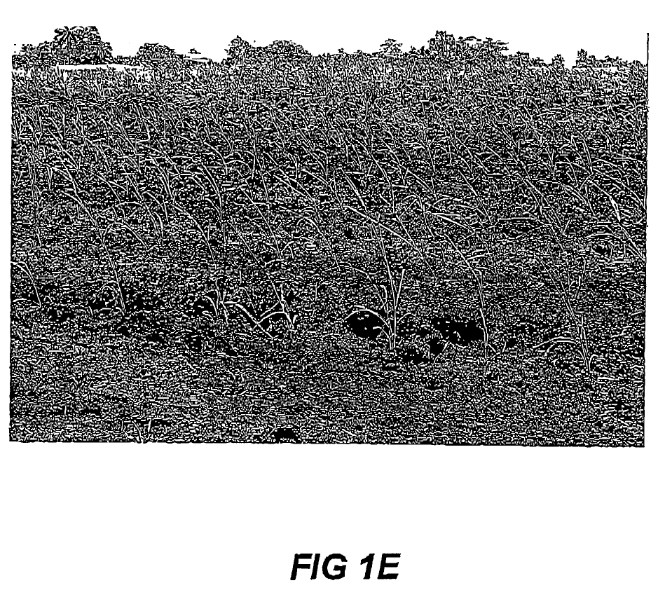

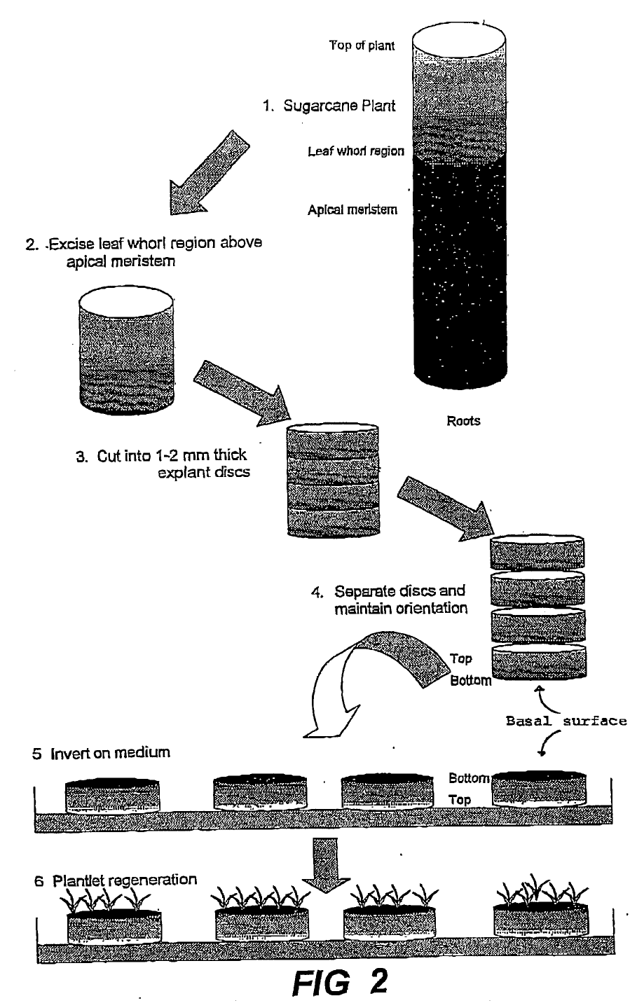

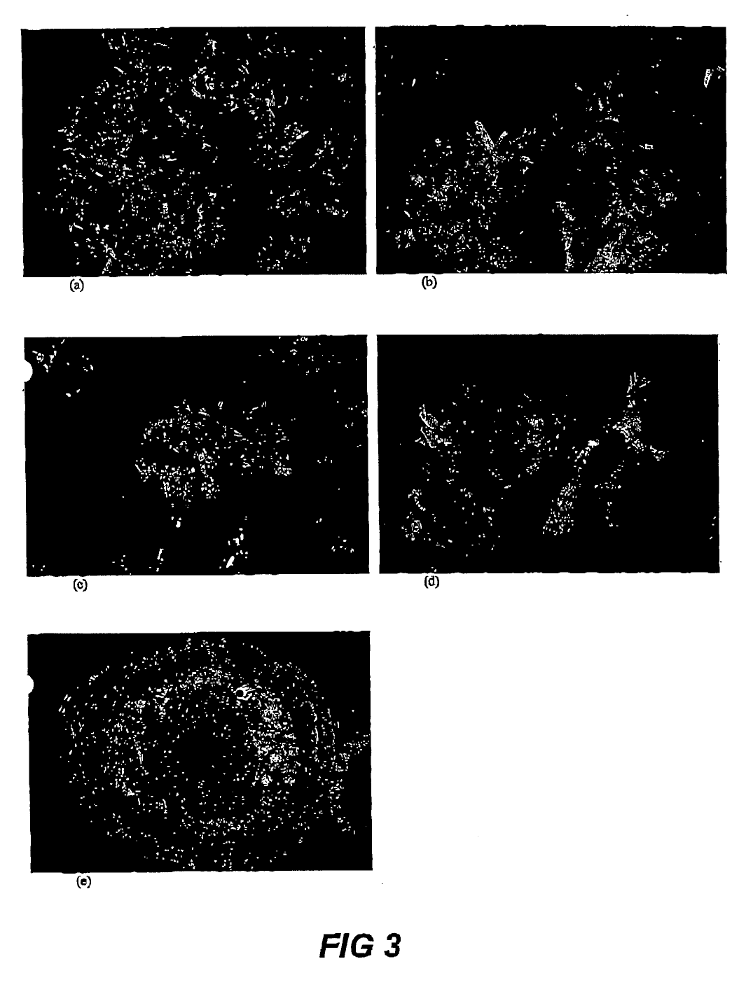

FIG. 1 shows an example of shoot generation from thin sections of sugarcane leaf spindle, and FIG. 2 provides a schematic representation showing explant orientation during the sectioning and culture processes. FIG. 3 shows an example of shoot generation from thin sections of sugarcane inflorescence.

### EXAMPLE 2

### Influence of Explant Thickness and Orientation On Shoot Generation

Leaf spindle explants of sugarcane cultivar Q165 were cultured on solid MS medium/agar in the presence of 4 μM BA and 10 μM NAA. The explants differed in thickness (1-2 mm versus 5-6 mm), orientation of explant (basal surface contacting medium versus apical surface contacting medium) and duration of culture. Table 1 reports results after 5 weeks of culture, Table 2 after 6 weeks of culture, and Table 3 after 8 weeks of culture.

It was clear that a much higher percentage of explants having their basal surface not contacting the medium (“top down”) produced shoots. This is particularly evident in Table 3, where the frequency of 1.0-2.0 mm explants producing shoots was up to 28-fold greater in the “top down” orientation. Also, the only explants which produced large numbers of shoots (>20 per explant) after 6 or 8 weeks of culture were those where the explant was oriented so that the basal surface did not contact the medium (“top down”). This trend was also evident, although less marked, after 5 weeks of culture.

Significantly more shoots per explant were produced where the explant was “top down” and 1-2 mm in thickness rather than 5-6 mm in thickness.

It should also be noted that plant regeneration occurred only at the basal surface of the explants. Thick sections (>10 mm in thickness) placed horizontally with the basal surface not contacting the medium preferentially generated shoots from the basal surface.

It has therefore been shown that shoot generation preferentially occurs from the basal surface. This conclusion holds for solid and liquid culture media.

### EXAMPLE 3

### Influence of Explant Sections On Shoot Generation In Cultivars Q165 and 90N876

From the preceding experimental data, it has been concluded that shoot growth occurs preferentially from the basal surface of explants rather than the apical surface. Furthermore, a dramatic increase in shoot regeneration frequency occured when the basal surface of the explant was oriented so as not to be in contact with the culture medium.

The data set forth in Tables 4-6 further demonstrate preferential shoot growth from thin sections taken from the basal and middle regions, compared to the apical regions, of sugarcane leaf spindle. Also, the concentrations of BA and NAA were varied in order to determine their influence upon shoot generation. In all cases, the basal surface of each section (whether a basal, middle or apical section) was not in contact with the culture medium. The percentage of explants producing shoots were scored, as were the number of shoots produced per explant. By both criteria, the basal and middle sections generally produced more shoots and with greater frequency than a similarly oriented apical section. This phenomenon was demonstrable in both sugarcane cultivars tested, and was largely independent of BA or NAA concentration.

### EXAMPLE 4

### Influence Of Different Cytokinin And Auxin Concentrations On Shoot Generation In Cultivar Q165

The effect of different concentrations of NAA, and either BA or KIN, was tested on shoot regeneration from 1-2 mm or 5-6 mm TS leaf explants from the Q165 sugarcane cultivar. The results are shown in Tables 7-10.

Generally, as is particularly evident from Tables 7, 8 and 10, 4 μM BA or KIN were optimal at any given concentration of NAA. From the data in Tables 7-9, there was no noticeable trend in terms of NAA concentration on either the percentage of explants producing shoots, or the number of shoots produced per explant.

### EXAMPLE 5

### Influence Of Different NAA Concentrations On Shoot Generation In cultivars Q179, 90N876 and 91N406

A comparison of the effect of different NAA concentrations was performed using different sugarcane cultivars. The data are shown in Tables 11-13.

Shoot regeneration from TS explants of sugarcane cultivar Q179 was relatively unaffected by varying the concentration of NAA between 10 and 40 μM. In contrast, the percentage of 90N876 explants producing shoots occurred at the highest concentration (40 μM) of NAA tested. This was true also with regard to production of large numbers of shoots (>20 per explant). Furthermore, at the lowest concentration of NAA tested (10 μM), all shoot-producing explants produced small numbers of shoots (1-10 per explant). A similar trend was evident with 91N406 explants, the optimal concentration of NAA being 40 μM.

### EXAMPLE 6

### Influence Of Different Cytokinin And Auxin Concentrations On Shoot Generation In Cultivars Q187 and Q124

Cross-testing of NAA and BA concentrations was performed using TS explants obtained from the abovementioned sugarcane cultivars. The data are shown in Tables 14 and 15. In Q187, 4 μM BA was clearly more efficient than 8 μM BA in terms of the percentage of explants producing shoots. This trend was less evident with regard to the number of shoots produced, although only in the presence of 4 μM BA did any explants produce more than 10 shoots.

The Q124 data in Table 15 are very different to the Q187 data in Table 14. There was no clear preference for 4 μM BA over 8 μM BA by any of the regeneration criteria examined. However, higher concentrations of NAA (40 μM) promoted a slightly higher percentage of explants producing shoots. There was no clear trend in terms of the number of shoots produced with regard to either NAA or BA concentration.

### EXAMPLE 7

### Shoot Generation From Explants Obtained From Cultivars Q57 and Q117

Table 16 demonstrates shoot regeneration from explants obtained from sugarcane cultivars Q57 and Q117. As is the case in all other experiments reported herein, a cytokinin and auxin is crucial to shoot regeneration from explants.

### EXAMPLE 8

### Field Trials to Assess Somaclonal Variation

A field trial was established at Bureau of Sugar Experiment Station, Gordonvale, Meringa Q 4865 to test whether plants regenerated using the tissue culture system exhibit somaclonal variation when grown in the field. This was-tested by planting tissue culture-derived plants germinated from single eye setts in a replicated blocked design and measuring a range of morphological, agronomic and biochemical parameters from both types of plants for comparison.

13.1 Statistical Design

Randomised block design of six (6) replicates×two (2) clones (Q96 and Q 117)×two (2) treatments (propagule origin—one-eye sett vs tissue culture propagules).

13.2 Plotformat

Three rows at 1.5 m×19 propagules at 0.5 m spacing, with 1.0 m spacing between plots. Measurements were taken on the innermost 13 stools in the middle row.

13.3. Statistical Analysis

The format for the analysis of variance for data collected in a single environment is as follows:

| Source of variation | Degrees of freedom | d.f. |
|---|---|---|
| Replicates | (r-1) | 5 |
| Clones | (c-1) | 1 |
| Treatments | (t-1) | 1 |
| Clones × Treatments | (c-1)(t-1) | 1 |
| Error | (r-1)(ct-1) | 15 |
| Sampling | rct(s-1) | 288 |
| Total | rcts-1 | 311 |

13.4 Traits

The following traits are suggested for measurement as they encompass a diverse range of developmental and physiological processes. Final measurements will be conducted in a 12-month-old crop.

| Measurement basis | Trait |
|---|---|
| Stool | No. culms per stool |
| | Percent flowered culms |
| | Juice Brix |
| | Percent fibre (fwb) |
| | Percent moisture (fwb) |
| | Juice pol. reading |
| Stool mean | Culm diameter |
| | No. nodes per culm (last exposed |
| | dewlap to ground) |
| | Culm length (last exposed dewlap |
| | to ground) |
| | Length of last exposed dewlap leaf |
| | Width of last exposed dewlap leaf |

13.5 First Assessment

The number of tillers in each of the central 13 plants in the middle row of each plot was counted. These data were analysed as a replicated two-factor design with sampling.

13.6 Results and Discussion

The complete analysis of variance is presented in Table 17. The main effect of clones was highly significant. The effect due to propagation method and the interaction between cultivar and propagation method were not significant. The CV% values for this analysis was high (64.7%), indicating high plot to plot variation. As this is an early crop measurement the high error may reflect variation within the main effects, particularly propagation method, that has not yet equilibrated. Such variation may have arise during establishment of the propagules or during growth of the propagules on the benches, i.e., influences prior to the establishment of the field trial. If this is true, CV% values will decline in subsequent counts.

The plot to plot error (δ²ₛ+13δ²ₛ) was highly significant relative to the sampling error (δ²ₛ) (Table 17). The error ratio test of 13δ²ₑ/δ²ₛ (=(F_{sub-sampling}−1)=4.1) indicates the sub-sampling strategy of using 13 stools per plot to determine the mean tillers per stool in a plot is adequate. This value exceeds the rule of thumb threshold of 3.0 for this test. The mean numbers of tillers per stool for Q96 and Q117 were 7.5 and 5.0, respectively.

In conclusion, there were major differences between cultivars for number of tillers per stool. There was no difference between the propagation methods, and there was no interaction between the main effects of cultivars and propagation method. Clearly, the micropropagation method of the invention produces a population of mature sugarcane plants without any evident somaclonal variation.

### EXAMPLE 9

### Regeneration From inflorescence Thin Sections

Experiments to test regeneration from 2-3 mm thin sections of inflorescence were performed and the data summarized in Table 18 and FIG. 3. In Table 18, orientation of the main floral axis (isolated from the inflorescence section) so that the basal surface was not in contact with the culture medium (“top down”) produced the highest percentage of regenerating explants (see a). When sections were placed laterally so that neither the basal surface or the apical surface was in contact with the culture medium, regeneration occurred best in the presence of 4 μM BA and 40 μM NAA.

It is noted that in (c), culture of immature floral tissue (rachis branches) dissociated from the remainder of the thin section resulted in no regeneration at all.

Referring to (d), it is clear that thin sections of intact inflorescence oriented “top down” produced the highest frequency of regeneration and the highest frequency of shoots per regenerating explant.

Of particular note is that regeneration from inflorescence tissue was predominantly embryogenic rather than organogenic, since the emergence of roots was accompanied by emergence of shoots. Furthermore, in general regeneration was rapid and did not occur via a lengthy callus phase, with the resultant expectation that somaclonal variation should be minimized by this technique.

### EXAMPLE 10

### Sonmatic Embryogenesis In Sugarcane

The realization that sugarcane regeneration could occur via an embryogenic mechanism that does not involve transitional callus formation, led the present inventors to investigate sugarcane somatic embryogenesis in more detail. Embryogenic regeneration is particularly useful in combination with transformation, as transformed, embryogenically-regenerated plants are less likely to display chimerism.

These experiments were performed essentially as hereinbefore described except that the auxin chlorophenoxyacetic acid (CPA) and 3 amino-2,5-dichlorobenzoic acid AD were investigated as well as NAA and BA. All TS explants were 1.0-2.0 mm thick leaf whorl sections oriented “top down” to maximize regeneration efficiency.

Referring to Table 19, CPA up to 10 μM did not induce any considerable level of callus development, but instead tended to develop somatic embryos directly. However, application of 20 μM and 40 μM CPA caused profuse callus production. This callus may or may not produce embryos.

To produce the data shown in Table 20, all the cultures were maintained under light (16 hr/day) from the start of the experiment, otherwise conditions were as for the experiments reported in Table 19. Provision of light inhibited callus production substantially. Although callus production was noticed at 20 μM and 40 μM levels of CPA, it was not to the level observed in cultures maintained in the dark, as reported in Table 19.

Referring now to Table 21, shoot regeneration via embryogenesis was apparent with no growth abnormalities, for BA 4 μM, NAA 10 μM and CPA 5-10 μM. Any further increase in CPA would be expected to cause considerable growth inhibition.

As shown in Table 22, the effect of AD was similar to that of CPA, except that CPA cultures produced more healthy looking plants.

Table 23 reports data from an experiment similar to that reported in Table 21, the only difference being the use of a different sugarcane cultivar, 85C542. In Tables 24 and 25, the data were obtained in respect of cultivar, 85C542, and CPA is replaced with 3 amino-2,5-dichlorobenzoic acid (AD). Clearly regeneration is favoured when cultures were maintained in dark initially rather than in light.

CPA and AD were the most effective inducers of high frequency somatic embryogenesis in cultures of leaf sections. CPA is the preferred inducer as it is more successful in developing somatic embryos at high frequency than AD. Furthermore, CPA did not cause any morphological abnormalities in the regenerated plants whereas AD developed albinos occasionally. So, clearly CPA is the preferred auxin for sugarcane embryogenesis.

### EXAMPLE 11

### Direct Regeneration Of Sorghum (Sorghum bicolor L.)

Thin section culture was undertaken in the commercially important sorghum variety New Nugget. With this methodology the present inventors have produced numerous plants from a single originating plant within three months. Plantlets produced from regenerated transferred to the glasshouse produced plants were fertile and produced viable seeds. Tissue culture-derived plants were morphologically identical to those produced from seeds.

11.1 Methodology

Young developing leaf sheaths (the innermost 3-4 whorls at the shoot tip) were used as the explant. Explants were preferably 1.0-2.0 mm thick transverse sections taken from the whorls. MS nutrient medium containing 3% sucrose and 7.5 g/L Davis J3 agar was used as the basal medium for all experiments. MS-based medium with several plant growth regulators was tested as a micropropagation medium. Shoot regeneration and somatic embryo formation was observed in media containing chlorophenoxyacetic acid (CPA) alone or in combination with benzylaminopurine (BA).

11.2 Results

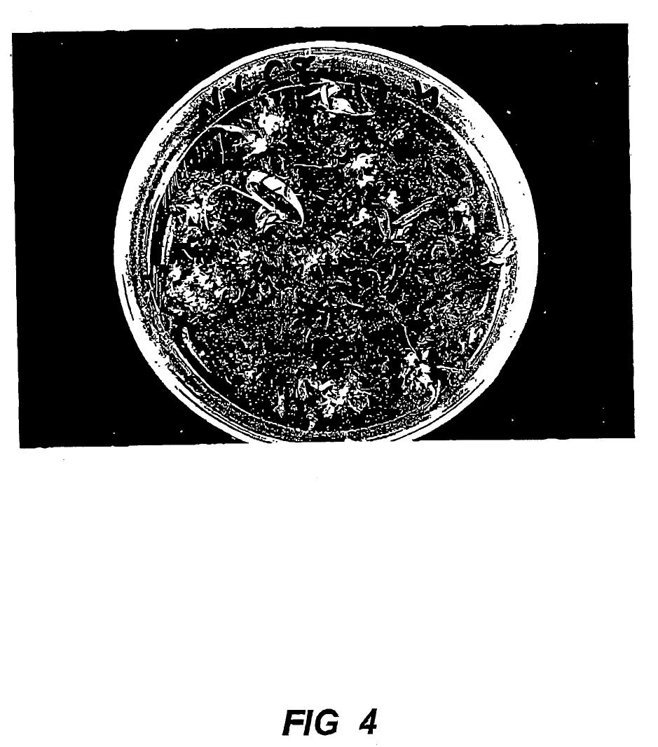

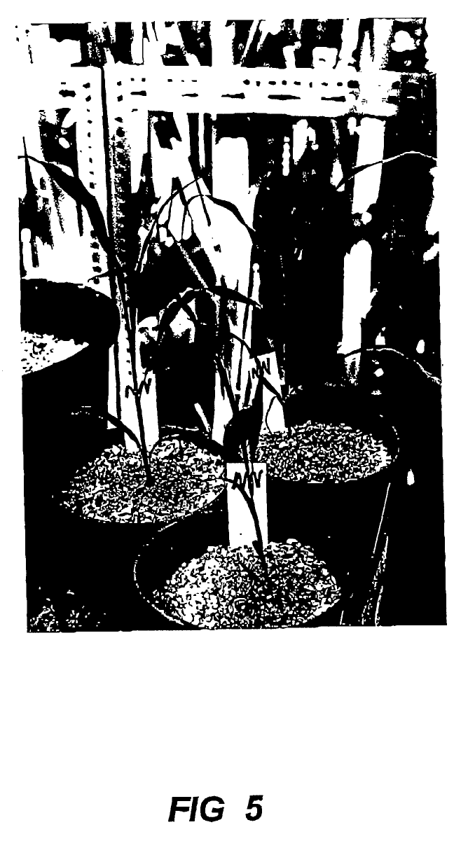

The production of regenerating tissue and sorghum plants derived therefrom are shown in FIGS. 4 and 5 respectively.

Shoot regeneration and somatic embryo formation was evident as early as 2-3 weeks in CPA supplemented media.

Shoots and plantlets were formed from embryos when they were placed onto basal MS without CPA. Shoot regeneration was enhanced by the addition of kinetin after the initial embryo formation. Shoots readily formed roots on basal MS medium.

The time from culture initiation to plants in greenhouse was approximately 10-12 weeks.

Referring to Table 26, CPA at a concentration in the culture medium between 4 μM and 25 μM was efficacious, although 8 μM CPA appeared to be optimal for the direct regeneration of shoots and somatic embryos.

It was noted that under these conditions, especially if cultures were maintained for longer periods on CPA or if higher CPA concentrations were used, a white friable embryogenic callus was also produced. This callus also produced shoots when placed on basal MS or MS supplemented with kinetin.

Experiments using the phytohormones (BA;4 μM and NAA; 10-40 μM) which had been optimised for the sugarcane were performed. None of the cultures showed any sign of shoot development. Experiments with other hormone combinations, such as utilizing dicamba, picloram and thidiazuron, were also unsucessful although some callus growth was seen on cultures containing dicamba.

In experiments incorporating CPA (0-40 μM) and CPA with BA (0 or 2.5 μM) combinations, after three weeks of culture, embryo-like structures could be clearly seen in cultures with several levels of CPA (Table 27). After four weeks some of these cultures were placed onto basal MS and began to develop some shoots. These shoots have developed into rooted plants. The remaining cultures were placed onto a medium with a reduced level of CPA (1-2 μM) and 2 μM kinetin. Within one week of transfer shoots started to appear from both embryos and callus.

11.3 Discussion

The implications of the sorghum experiments show that plants can be directly regenerated from leaf explants without passing through callus, such that the plants produced by this method can be used for rapid mass propagation of sorghum, if required. Production of transgenic sorghum plants using this method may be suitable for both biolistic and Agrobacterium-mediated genetic transformation with substantially reduced the time required to produce transgenic plants. Furthermore. somaclonal variation is likely to be minimised.

### EXAMPLE 12

### Regeneration Of Wheat

A number of plants from individual thin transverse sections (approximately 6) have been regenerated. Successful regeneration of “shoots” or “plants” was achieved on media containing between 5 μM and 40 μM chlorophenoxy-acetic acid (CPA) with or without an additional time on media with 5 μM zeatin, as shown in Table 28.

12.1 Methodology

Young wheat plants 15-40 cm tall were used as starting material. Transverse excisions were made to produce thin sections (up to 2.0 mm) of leaf tissue which sometimes included immature developing floral material depending on where the sections were taken along the stem. In some cases, the sections were from regions of hollow “stem”.

Thin sections were placed with the apical surface in contact with the MS medium (“top down”) containing phytohormones for high frequency regeneration, namely 5 to 10 μM CPA for three to four weeks. These were then transferred to 5 μM zeatin for further culturing.

After the appearance of small shoots and/or plantlets, these were left to develop on media containing 5 μM zeatin. When these had attained sufficient size (approximately greater than 20 mm) they were excised and transferred to fresh media. Plantlets were transferred to ½ strength MS media when capable of growth in the absence of zeatin.

12.2 Results

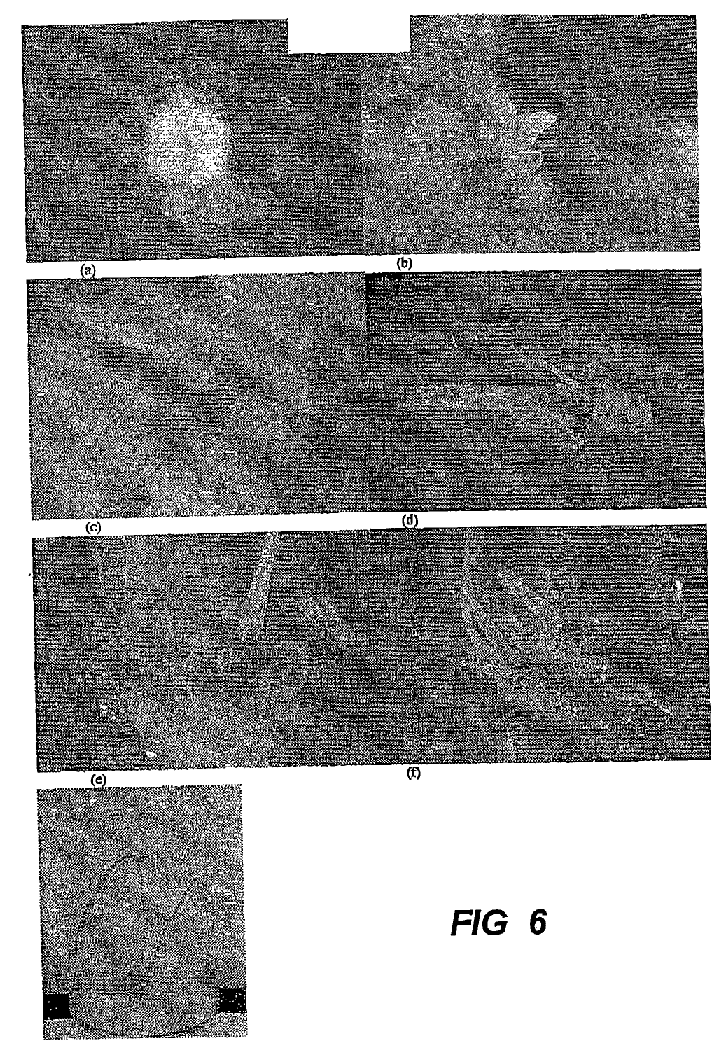

Regeneration was direct from the explants and usually from around the outer leafwhorl and thus did not require a lengthy passage through callus culture (FIG. 6).

Shoot development was only seen in treatments where wheat explants were placed with the apical side in contact with the MS media containing phytohormones (“top down”). No shoots developed in the treatment where explants were placed apical side up (Table 28). Thus there is a requirement that the apical side of the thin transverse sections be in contact with the media. Put another way, regeneration was optimal when the basal surface of the wheat explant is oriented so as to be not in contact with the culture medium.

Regeneration of shoots/plants occurred directly without a lengthy passage though callus culture. Regeneration appeared to be predominantly of an organogenic type although it is contemplated that embryogenic regeneration will be possible by manipulating the phytohormone conditions.

### EXAMPLE 13

### Conclusion

In conclusion, regeneration from leaf spindle and inflorescence thin sections is greatly influenced by explant orientation. In contrast to the prior art, the optimal orientation is such that a basal surface of the explant is not in contact with the culture medium. The present invention demonstrates regeneration from sugarcane, sorghum and wheat thin section explants, wherein transitional callus formation is minimized or virtually eliminated. Furthermore, the present inventors show that regeneration is affected by the type and concentration of auxin and/or cytokinin present during culture. In at least sugarcane, this also affects the type of regeneration, that is whether regeneration is organogenic or embryogenic. The present invention therefore provides a highly efficient non-callus regeneration system applicable to any monocot, and thereby provides a system that avoids or reduces problems associated with callus-based regeneration, such as somaclonal variation.

It will be appreciated that the invention is not limited to that which is described in detail herein, and that a variety of embodiments and modifications are contemplated which nevertheless fall within the broad spirit and scope of the invention.

All computer programs, scientific literature and patent literature referred to in this specification are incorporated herein by reference.

TABLE 1

| | | | % of morphogenic explants | | |
|---|---|---|---|---|---|
| | | % of explants | producing 1-10 (+), 11-20 (++) | | |
| Explant | | producing | and >20 (+++) shoots | | |
| Size | Orientation | shoots | + | ++ | +++ |
| 1-2 mm | Top up | 7.8 | 100 | 0.0 | 0.0 |
| 1-2 mm | Top down | 79.0 | 18.7 | 26.5 | 54.8 |
| 5-6 mm | Top up | 41.7 | 40.0 | 48.6 | 11.4 |
| 5-6 mm | Top down | 54.6 | 30.5 | 32.2 | 37.3 |

TABLE 2

| | | | % of morphogenic explants | | |
|---|---|---|---|---|---|
| | | % of explants | producing 1-10 (+), 11-20 (++) | | |
| Explant | | producing | and >20 (+++) shoots | | |
| Size | Orientation | shoots | + | ++ | +++ |
| 1-2 mm | Top up | 2.6 | 100 | 0.0 | 0.0 |
| 1-2 mm | Top down | 87.5 | 12.3 | 25.7 | 62.0 |
| 5-6 mm | Top up | 14.0 | 28.6 | 71.4 | 0.0 |
| 5-6 mm | Top down | 83.6 | 13.0 | 30.4 | 56.6 |

TABLE 3

| | | | % of morphogenic explants | | |
|---|---|---|---|---|---|
| | | % of explants | producing 1-10 (+), 11-20 (++) | | |
| Explant | | producing | and >20 (+++) shoots | | |
| Size | Orientation | shoots | + | ++ | +++ |
| 1-2 mm | Top up | 3.0 | 100 | 0.0 | 0.0 |
| 1-2 mm | Top down | 88.1 | 11.3 | 16.5 | 77.6 |
| 5-6 mm | Top up | 24.0 | 33.3 | 66.7 | 0.0 |
| 5-6 mm | Top down | 96.0 | 50.0 | 33.3 | 16.7 |

TABLE 4

| | | | | % of morphogenic explants | | |
|---|---|---|---|---|---|---|
| Medium | | | % of explants | producing 1-10 (+), 11-20 (++) | | |
| BA | NAA | Explant | producing | and >20 (+++) shoots | | |
| (μM) | (μM) | position | shoots | + | ++ | +++ |
| 4 | 5 | Basal | 77.5 | 19.4 | 41.9 | 38.7 |
| 4 | 5 | Middle | 85.0 | 11.8 | 32.4 | 55.8 |
| 4 | 5 | Apical | 16.7 | 100 | 0.0 | 0.0 |
| 4 | 10 | Basal | 64.0 | 15.6 | 46.8 | 37.6 |
| 4 | 10 | Middle | 74.0 | 13.5 | 27.0 | 59.5 |
| 4 | 10 | Apical | 60.0 | 33.3 | 40.0 | 26.7 |
| 4 | 20 | Basal | 86.6 | 7.7 | 19.2 | 73.1 |
| 4 | 20 | Middle | 67.5 | 7.4 | 25.9 | 66.7 |
| 4 | 20 | Apical | 30.0 | 11.1 | 33.3 | 55.6 |
| 8 | 5 | Basal | 72.5 | 37.9 | 41.4 | 20.7 |
| 8 | 5 | Middle | 77.5 | 19.4 | 48.3 | 32.3 |
| 8 | 5 | Apical | 32.5 | 61.5 | 38.5 | 0.0 |
| 8 | 10 | Basal | 47.5 | 26.3 | 57.9 | 15.8 |
| 8 | 10 | Middle | 67.5 | 22.2 | 25.9 | 51.9 |
| 8 | 10 | Apical | 15.0 | 0.0 | 66.7 | 33.3 |
| 8 | 20 | Basal | 65 | 7.7 | 15.4 | 76.9 |
| 8 | 20 | Middle | 85 | 14.7 | 26.5 | 58.8 |
| 8 | 20 | Apical | 12.5 | 20.0 | 80.0 | 0.0 |
| 12 | 10 | Basal | 67.5 | 18.5 | 29.6 | 51.9 |
| 12 | 10 | Middle | 97.5 | 12.8 | 25.6 | 61.6 |
| 12 | 10 | Apical | 32.5 | 61.5 | 15.4 | 23.1 |
| 12 | 20 | Basal | 84.0 | 4.8 | 11.9 | 83.3 |
| 12 | 20 | Middle | 70.0 | 5.7 | 14.3 | 80.0 |
| 12 | 20 | Apical | 20.0 | 50.0 | 30.0 | 20.0 |

TABLE 5

| | | | | % of morphogenic explants | | |
|---|---|---|---|---|---|---|
| Medium | | | % of explants | producing 1-10 (+), 11-20 (++) | | |
| BA | NAA | Explant | producing | and >20 (+++) shoots | | |
| (μM) | (μM) | position | shoots | + | ++ | +++ |

| | | | | | | |
|---|---|---|---|---|---|---|
| 4 | 10 | Basal | 90.0 | 0.0 | 30.6 | 69.4 |
| 4 | 10 | Middle | 82.5 | 3.0 | 18.0 | 79.0 |
| 4 | 10 | Apical | 55.0 | 27.3 | 40.9 | 31.8 |
| 4 | 20 | Basal | 85.0 | 0.0 | 17.6 | 82.4 |
| 4 | 20 | Middle | 80.0 | 6.3 | 21.9 | 71.8 |
| 4 | 20 | Apical | 60.0 | 16.7 | 36.7 | 46.6 |
| 4 | 40 | Basal | 83.3 | 0.0 | 24.0 | 76.0 |
| 4 | 40 | Middle | 85.0 | 8.8 | 29.4 | 61.8 |
| 4 | 40 | Apical | 75.0 | 10.0 | 40.0 | 50.0 |

TABLE 6

| | | % of explants | | | |
|---|---|---|---|---|---|
| Medium | | producing shoots | | | Frequency of shoot |
| BA (μM) | NAA (μM) | Basal | Middle | Apical | regeneration |
| 0 | 0 | 0 | 0 | 0 | 0 |
| 4 | 10 | 4 | 2 | 0 | Most of the |
| 4 | 20 | 24 | 58 | 46 | regenerating |
| 4 | 30 | 28 | 86 | 38 | explants produced |
| 4 | 40 | 54 | 92 | 35 | one or two clusters |
| 8 | 10 | 0 | 35 | 10 | of shoots (2-10 |
| 8 | 20 | 20 | 62 | 20 | shoots in each |
| 8 | 30 | 17 | 16 | 18 | cluster). However, |
| 8 | 40 | 25 | 58 | 67 | occasionally some |
| | | | | | explants in B₄N₃₀ |
| | | | | | and B₄N₄₀ media |
| | | | | | regenerated large |
| | | | | | numbers (>50/ |
| | | | | | explant) of shoots. |

TABLE 7

| | | | | % of morphogenic explants | | |
|---|---|---|---|---|---|---|
| Medium | | | % of explants | producing 1-10 (+), 11-20 | | |
| BA | KIN | NAA | producing | (++) and >20 (+++) shoots | | |
| (μM) | (μM) | (μM) | shoots | + | ++ | +++ |
| 0 | 0 | 0 | 0.0 | 0.0 | 0.0 | 0.0 |
| 4 | 0 | 10 | 45.5 | 25.3 | 37.4 | 37.3 |
| 4 | 0 | 20 | 55.7 | 18.8 | 35.9 | 45.3 |
| 4 | 0 | 40 | 56.5 | 29.2 | 31.0 | 39.8 |
| 0 | 4 | 10 | 59.3 | 54.0 | 39.3 | 6.7 |
| 0 | 4 | 20 | 75.9 | 39.5 | 33.3 | 27.2 |
| 0 | 4 | 40 | 66.6 | 25.0 | 45.0 | 30.0 |

TABLE 8

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 94.4 | 3.5 | 9.4 | 87.1 |
| 4 | 20 | 85.5 | 5.2 | 9.6 | 85.2 |
| 4 | 40 | 80.8 | 8.2 | 13.4 | 78.4 |
| 4 | 60 | 92.5 | 0.0 | 8.1 | 91.9 |
| 8 | 10 | 84.4 | 5.0 | 17.1 | 77.6 |
| 8 | 20 | 86.6 | 9.0 | 21.8 | 69.2 |
| 8 | 40 | 85.0 | 3.9 | 19.6 | 76.5 |
| 8 | 60 | 89.2 | 8.4 | 17.8 | 73.8 |

TABLE 9

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 88.1 | 11.3 | 16.5 | 72.2 |
| 4 | 20 | 81.9 | 1.5 | 13.7 | 84.8 |
| 4 | 40 | 88.3 | 5.7 | 16.7 | 77.6 |

TABLE 10

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 96 | 29.1 | 33.3 | 37.6 |
| 4 | 20 | 92 | 21.7 | 47.8 | 30.5 |
| 4 | 40 | 96 | 37.5 | 41.7 | 20.8 |
| 8 | 10 | 84 | 47.6 | 9.5 | 42.9 |
| 8 | 20 | 80 | 30.0 | 50.0 | 20.0 |
| 8 | 40 | 90 | 18.5 | 37.0 | 44.5 |

TABLE 11

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 2.0 | 0.0 | 100 | 0.0 |
| 4 | 20 | 20.0 | 40.0 | 45.0 | 15.0 |
| 4 | 30 | 15.0 | 46.7 | 40.0 | 13.3 |
| 4 | 40 | 0.0 | 0.0 | 0.0 | 0.0 |

TABLE 12

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 15.0 | 100 | 0.0 | 0.0 |
| 4 | 20 | 34.3 | 62.5 | 26.0 | 11.5 |
| 4 | 40 | 76.0 | 28.9 | 31.6 | 39.5 |

TABLE 13

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 10.0 | 0.0 | 100 | 0.0 |
| 4 | 20 | 27.1 | 63.2 | 21.1 | 15.7 |
| 4 | 30 | 30.0 | 58.3 | 33.3 | 8.4 |
| 4 | 40 | 45.0 | 52.8 | 22.2 | 25.0 |

TABLE 14

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 3.8 | 100 | 0.0 | 0.0 |
| 4 | 20 | 29.2 | 88.6 | 11.4 | 0.0 |
| 4 | 30 | 15.0 | 87.5 | 12.5 | 0.0 |
| 4 | 40 | 46.4 | 64.7 | 17.7 | 17.6 |
| 8 | 10 | 0.0 | 0.0 | 0.0 | 0.0 |
| 8 | 20 | 5.8 | 100 | 0.0 | 0.0 |
| 8 | 40 | 4.6 | 100 | 0.0 | 0.0 |

TABLE 15

| | | | % of morphogenic explants | | |
|---|---|---|---|---|---|
| | | % of explants | producing 1-10 (+), 11-20 | | |
| Medium | | producing | (++) and >20 (+++) shoots | | |
| BA (μM) | NAA (μM) | shoots | + | ++ | +++ |
| 0 | 0 | 0.0 | 0.0 | 0.0 | 0.0 |
| 4 | 10 | 80 | 21 | 20 | 59 |
| 4 | 20 | 74 | 74 | 29 | 58 |
| 4 | 30 | 97 | 10 | 21 | 69 |
| 4 | 40 | 93 | 16 | 21 | 63 |
| 8 | 10 | 85 | 18 | 29 | 53 |
| 8 | 20 | 78 | 10 | 18 | 72 |
| 8 | 30 | 97 | 23 | 26 | 51 |
| 8 | 40 | 93 | 14 | 32 | 54 |

TABLE 16

| | | | | % of morphogenic explants | | |
|---|---|---|---|---|---|---|
| | Medium | | % of explants | producing 1-10 (+), 11-20 | | |
| | BA | NAA | producing | (++) and >20 (+++) shoots | | |
| Cultivar | (μM) | (μM) | shoots | + | ++ | +++ |
| Q57 | 0 | 0 | 0.0 | 0.0 | 0.0 | 0.0 |
| | 4 | 20 | 23.3 | 31.4 | 68.6 | 0.0 |
| Q117 | 0 | 0 | 0.0 | 0.0 | 0.0 | 0.0 |
| | 4 | 10 | 60.0 | 16.7 | 21.4 | 61.9 |

TABLE 17

| | | Sum of | | |
|---|---|---|---|---|
| Source of variation | d.f. | squares | Mean square | ‘F’ |
| Replicates | 5 | 107.641 | 21.528 | 1.307 |
| Clones (C) | 1 | 487.500 | 487.500 | 29.599 |
| Propagation method (P) | 1 | 6.205 | 6.205 | 0.376 |
| C × P | 1 | 8.667 | 8.667 | 0.537 |
| Error | 15 | 247.051 | 16.470 | 5.080 |
| Sub-sampling¹ | 286 | 927.231 | 3.242 | |
| Total | 309 | 1,784.295 | | |

TABLE 18

| | | | Phytohormone | Number of explants | total no. | Percentage of | ^{A}Frequency of shoots |
|---|---|---|---|---|---|---|---|
| Explant | | CV | Treatment (μM) | showing reg'n | of explants | explants regenerating | on regenerating explants |
| (a) | thin pieces | Q152 | B4N40 | 0 | 32 | 0 | |
| | of main floral axis | Q165 | B4N10 | 8 | 72 | 11 | * |
| | | Q152 | B4N10 | 8 | 16 | 50 | * |
| | | | (top down) | | | | |
| (b) | ˜8 mm pieces | Q152 | B4N40 | 45 | 136 | 38 | ** |
| | of main floral axis | | B4N10 | 7 | 32 | 22 | * |
| | placed laterally | | | | | | |
| | | | B4N60 | 1 | 40 | 2.5 | * |
| | | | 2,4-D14 | 0 | 46 | 0 | |
| | | Q165 | B4N10 | 4 | 70 | 6 | |
| (c) | immature floral | Q165 | B4N10 | 0 | 10 | 0 | |
| | tissues surrounding | | B4N10 | 0 | 15 | 0 | |
| | main stem spread on | | | | | | |
| | medium | | | | | | |
| (d) | thin sections of | Q165 | B4N10 | 80 | 83 | 96 | *** |
| | floral material | | (top down) | | | | |

TABLE 19

| | | % of explants producing | | |
|---|---|---|---|---|
| | | 1-10 (+), 11-20 (++), | | |
| Medium | % of explants producing | and >20 (+++) embryos/shoots | | |
| CPA (μM) | embryos/shoots | + | ++ | +++ |
| 0 | 0 | 0 | 0 | 0 |
| 5 | 70 | 35 | 62 | 3 |
| 10 | 89 | 18 | 39 | 43 |
| 20 | C | C | C | C |
| 40 | C | C | C | C |

TABLE 20

| | | % of explants producing | | |
|---|---|---|---|---|
| | | 1-10 (+), 11-20 (++), | | |
| Medium | % of explants producing | and >20 (+++) embryos/shoots | | |
| CPA (μM) | embryos/shoots | + | ++ | +++ |
| 0 | 0 | 0 | 0 | 0 |
| 5 | 8 | 100 | 0 | 0 |
| 10 | 47 | 89 | 11 | 0 |
| 20 | 22 | 92 | 8 | 0 |
| 40 | 13 | 88 | 12 | 0 |

TABLE 21

| | | | | % of explants producing | | |
|---|---|---|---|---|---|---|
| Medium | | | % of explants | 1-10 (+), 11-20 (++), and >20 | | |
| BA | NAA | CPA | producing | (+++) embryos/shoots | | |
| (μM) | (μM) | (μM) | embryos/shoots | + | ++ | +++ |
| 0 | 0 | 0 | 0 | 0 | 0 | 0 |
| 4 | 10 | 5 | 80 | 35 | 27 | 38 |
| 4 | 10 | 10 | 98 | 6 | 9 | 85 |
| 4 | 10 | 20 | 100 | 0 | 1 | 99 |
| 4 | 10 | 40 | 100 | 0 | 2 | 98 |

TABLE 22

| | | | | % of explants producing | | |
|---|---|---|---|---|---|---|
| Medium | | | % of explants | 1-10 (+), 11-20 (++), and >20 | | |
| BA | NAA | AD | producing | (+++) embryos/shoots | | |
| (μM) | (μM) | (μM) | embryos/shoots | + | ++ | +++ |
| 0 | 0 | 0 | 0 | 0 | 0 | 0 |
| 4 | 10 | 5 | 100 | 12 | 28 | 60 |
| 4 | 10 | 10 | 100 | 5 | 15 | 80 |
| 4 | 10 | 20 | 100 | 3 | 14 | 83 |
| 4 | 10 | 40 | 100 | 0 | 8 | 92 |

TABLE 23

| | | | | % of explants producing | | |
|---|---|---|---|---|---|---|
| Medium | | | % of explants | 1-10 (+), 11-20 (++), and >20 | | |
| BA | NAA | CPA | producing | (+++) embryos/shoots | | |
| (μM) | (μM) | (μM) | embryos/shoots | + | ++ | +++ |
| 0 | 0 | 0 | 0 | 0 | 0 | 0 |
| 4 | 10 | 10 | 98 | 13 | 24 | 53 |
| 4 | 10 | 20 | 100 | 5 | 15 | 80 |
| 4 | 10 | 40 | 86 | 17 | 21 | 52 |

TABLE 24

| | | | | % of explants producing | | |
|---|---|---|---|---|---|---|
| Medium | | | % of explants | 1-10 (+), 11-20 (++), and >20 | | |
| BA | NAA | AD | producing | (+++) embryos/shoots | | |
| (μM) | (μM) | (μM) | embryos/shoots | + | ++ | +++ |
| 0 | 0 | 0 | 0 | 0 | 0 | 0 |
| 4 | 10 | 10 | 12* | 100 | 0 | 0 |
| 4 | 10 | 20 | 18* | 100 | 0 | 0 |
| 4 | 10 | 40 | C | 0 | 0 | 0 |

TABLE 25

| | | | | % of explants producing | | |
|---|---|---|---|---|---|---|
| Medium | | | % of explants | 1-10 (+), 11-20 (++), and >20 | | |
| BA | NAA | AD | producing | (+++) embryos/shoots | | |
| (μM) | (μM) | (μM) | embryos/shoots | + | ++ | +++ |
| 0 | 0 | 0 | 0 | 0 | 0 | 0 |
| 4 | 10 | 10 | 69 | 54 | 17 | 29 |
| 4 | 10 | 20 | 73 | 57 | 27 | 16 |
| 4 | 10 | 40 | 64 | 56 | 22 | 22 |

TABLE 26

| | | % of explants producing | | |
|---|---|---|---|---|
| | | 1-10 (+), 11-20 (++), | | |
| Medium | % of explants producing | and >20 (+++) embryos/shoots | | |
| CPA (μM) | embryos/shoots | + | ++ | +++ |
| 0 | 0 | 0 | 0 | 0 |
| 4 | 60 | 28 | 56 | 16 |
| 8 | 90 | 25 | 31 | 44 |
| 16 | 80 | 31 | 33 | 37 |
| 25 | 90 | 39 | 27 | 34 |

TABLE 27

| | | | % of explants producing | | |
|---|---|---|---|---|---|
| | | % of explants | 1-10 (+), 11-20 (++), and > | | |
| Medium | | producing | 20 (+++) embryos/shoots | | |
| CPA (μM) | BA (μM) | embryos/shoots | + | ++ | +++ |
| 0 | 0 | 0 | 0 | 0 | 0 |
| 5 | 0 | 70 | 39 | 33 | 28 |
| 10 | 0 | 78 | 23 | 36 | 41 |
| 20 | 0 | 73 | 28 | 35 | 37 |
| 40 | 0 | 73 | 27 | 31 | 42 |
| 5 | 2.5 | 70 | 36 | 21 | 43 |

TABLE 28

| | |
|---|---|
| | Regeneration of shoots: * 15% |
| Phytohormone | explants with shoots, ** 15-40 |
| treatment (μM) | % explants with shoots |

(a) 12 weeks on designated phytohormone combination

| | |
|---|---|
| P5 | |
| P10 | |
| P20 | |
| P40 | |
| D5 | |
| D10 | |
| D20 | |
| D40 | |
| B4N1 | |
| B4N10 | |
| B4N20 | |
| B4N40 | |
| B0.1DP1 | |
| B1DP1 | |
| B5DP1 | |
| B10DP1 | |
| C5 | |
| C10 | * |
| C20 | * |
| C40 | |

(b) three weeks on first designated hormone combination then transfer

| | |
|---|---|
| C5→Z5 | * |
| C10→Z5 | ** |
| C20→Z5 | ** |
| C40→Z5 | * |
| P5→Z5 | |
| P10→Z5 | |
| P20→Z5 | |
| P40→Z5 | |

(c) thin transverse sections initiated apical up

C10
B4N20

1. A method of plant micropropagation including the step (i) of culturing an explant from a monocotyledonous plant in culture medium comprising a cytokinin and/or an auxin wherein a basal surface of said explant is oriented so as to be substantially not in contact with said medium during culture.

2. The method of claim 1, including the step (ii) of producing a plant shoot having a shoot meristem from the explant producd in step(i).

3. The method of claim 2, wherein step (ii) comprises:
(a) culturing the explant in the presence of a cytokin and/or auxin to produce plant shoots having meristems; and
(b) excising said plant shoots and culturing same to produce plantlets having roots.

4. The method of claim 3, including the step (iii) of propagating the plantlet obtained at step (ii) to produce a mature plant.

5. The method of claim 4, wherein micropropagation occurs without transition through a substantial callus phase.

6. The method of claim 5, wherein micropropagation occurs via embryogenesis.

7. The method of claim 5 wherein micropropagation occurs via organogenesis.

8. the method of claim 1, wherein the explant is a thin section (TS) explant.

9. The method of claim 8, wherein the TS explant is obtained from leaf spindle or whorl.

10. The method of claim 8, wherein the TS explant is obtained from inflorescence.

11. The method of claim 8, wherein the TS explant is 1.0-10.0 mm thick.

12. The method of claim 11, wherein the TS explant is of thickness selected from the group consisting of 1.0-2.0 mm, 2.0-3.0 mm and 5.0-6.0 mm.

13. The method of claim 1, wherein the monocotyledonous plant is of the Graminae family.

14. The method of claim 13, wherein the monocotyledonous plant is sugarcane.

15. The method of claim 13, wherein the monocotyledonous plant is a cereal.

16. The method of claim 15, wherein the cereal is wheat or sorghum.

17. The method of claim 1, wherein the cytokinin is selected from the group consisting of: kinetin (KIN), zeatin and 6-benzyladenine (BA).

18. The method of claim 1, wherein the auxin is selected from the group consisting of: α-napthaleneacetic acid (NAA), p-chlorophenoxyacetic acid (CPA) and 3 amino-2,5-dichlorobenzoic acid (AD).

19. The method of plant micropropagation including the steps:
(i) of culturing a TS explant of sugarcane leaf whorl or inflorescence in a culture medium comprising NAA and either BA or KIN, Wherein a basal surface of said explant is oriented so as to be substantially not in contact with said medium during culture; and
(ii) regenerating a sugarcane plant or plant tissue via organogenesis.

20. The method of claim 19, wherein NAA is present at a concentration in the range 10-60 μM.

21. The method of claim 19, wherein BA or KIN is present at a concentration in the range 4-12 μM.

22. The method of plant micropropagation including the steps:
(i) culturing a TS explant of sugarcane leaf whorl in culture medium comprising CPA or AD, wherein a basal surface of said explant is oriented so as to be substantially not in contact with said medium during culture; and
(ii) regenerating a sugarcane plant or plant tissue via embryongenesis.

23. The method of claim 22, wherein CPA is present alone at a concentration in the range 5-10 μM.

24. The method of claim 22, wherein CPA, BA and NAA are present in the culture medium.

25. The method of claim 22, wherein AD, BA and NAA are present in the culture medium.

26. A method of plant micropropagation including the step(i) of culturing a TS explant of sorghum leaf whorl in a culture medium comprising chlorophenoxyacetic acid (CPA) wherein a basal surface of said explant is oriented so as to be substantially not in contact with said medium during culture.

27. The method of claim 26 wherein chlorophenoxyacetic acid (CPA) is present at a concentration in the range 4-40 μM.

28. The method of claim 26 wherein BA is also present in the culture medium.

29. The method of claim 26, further including the step of culturing the TS explant in the presence of CPA and Kinetin (KIN) once regeneration has beeen initiated.

30. The method of claim 29, wherein the concentration of KIN is 2 μM and the concentration of CPA is 1-2 μM.

31. The method of plant micropropagation including the step (i) of culturing a TS explant of wheat leaf whorl in a culture medium comprising chlorophenoxyacetic acid (CPA) wherein a basal surface or said explant is oriented so as to be substantially not in contact with said medium during culture.

32. The method of claim 31 wherein chlorophenoxyacetic acid (CPA) is present at a concentration in the range 5-40 μM.

33. The method of claim 31, wherein further comprising the step of (ii) of replacing CPA with zeatin in the culture medium after regeneration has been initiated.

34. The method of claim 33, wherein zeatin is present in the culture medium at a concentration of 5 μM.

35. Regenerable plant tissue obtained according to the method of claim 1.

36. A plantlet obtained according to the method claim 1.

37. A monocotyledonous plant produced according to the method of claim 1.

38. A monocotyledonous plant of the Graminae family according to claim 37.

39. A sugarcane plant according to claim 38.

40. A cereal plant according to claim 38.

41. A sorghum or wheat plant according to claim 40.

42. Reproductive material derived from the monocotyledonous plant of any one of claims 37-41.

43. A seed according to claim 42.
